(19) United States
(12) Patent Application Publication
(10) Pub. No.: US 2010/0179229 A1
(43) Pub. Date: Jul. 15, 2010

(54) METHOD FOR MODULATING THE TASTE OF MATERIAL COMPOSITIONS CONTAINING AT LEAST ONE HIGH INTENSITY SWEETENER (HIS)

(75) Inventors: Markus Matuschek, Weinheim (DE); Martin B. Jager, Enkenbach-Alsenborn (DE); Alice Kleber, Bensheim (DE); Michael Krohn, Lorsch (DE); Holger Zinke, Zwingenberg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/602,189

(22) PCT Filed: Mar. 5, 2008
(86) PCT No.: PCT/EP2008/052657

§ 371 Date: Nov. 30, 2009

(51) Int. Cl. A61K 47/14; A61K 8/37; A61P 43/00; A61Q 90/00; A23L 1/236

(52) U.S. Cl. 514/785; 426/548

(57) ABSTRACT

Process for modulation of a taste and/or aftertaste in foods, articles consumed for pleasure, animal feeds, sweeteners and cosmetics comprising at least one high intensity sweetener HIS by addition of a taste modulator comprising at least one polyoxyethylene sorbitan fatty acid ester which is suitable for food, and use of a at least one polyoxyethylene sorbitan fatty acid ester which is suitable for food for taste modulation of foods, articles consumed for pleasure, animal feeds, sweeteners and cosmetics comprising at least one HIS.

### FIELD OF THE INVENTION

The present invention relates to a novel process for taste modulation, in particular for reduction of bitter taste and aftertaste, of compositions of matter which comprise at least one high intensity sweetener (HIS).

### PRIOR ART

Compositions of matter such as foods, drinks, articles consumed for pleasure, sweeteners, animal feeds, cosmetics and pharmaceuticals frequently comprise taste substances which are in principle unwanted or are too dominant or too low in the intensity in which they are present. In the sector of sweeteners, frequently, in addition to the sweet taste impressions, further taste impressions such as, for example, a metallic, chemical, bitter or synthetic taste or aftertaste occurs, which adversely affect the overall taste impression of the composition to be sweetened. In the context of the present invention, taste is taken to mean the immediate taste impression which is formed while the composition is situated in the mouth. Aftertaste is taken to mean the taste perception after swallowing, in particular after a waiting time of about 30 seconds.

For example, caffeine in tea or coffee, and also hop extracts in beer, are natural bitter substances which, however, in too high a concentration, cause an adverse taste impression. In special bitter drinks such as, for example, tonic water or bitter lemon, a characteristic bitter taste caused by the bitter substance quinine is desired to a particular extent. On the other hand, in particular the bitter taste or aftertaste of many artificial sweeteners such as, for example, acesulfame K (ACK) and saccharin is an unwanted offtaste of the sweetener in other lemonades. Fruit juices, in particular orange juice, also suffer from impairment of taste by, for example, flavonoid glycosides, which have a bitter taste. Many pharmaceutical active compounds, in particular ibuprofen, also have a strong bitter taste which leads to reduction in acceptance on intake of the active compound.

For reduction of the natural bitter taste of, for example, tea, coffee or orange juice, these foods, drinks, articles consumed for pleasure are either treated enzymatically in order to destroy the bitter-tasting substances, or the bitter substance, in the case of caffeine in tea or coffee, is removed by decaffeination.

Another possibility is to enclose the bitter-tasting active compound by formulation techniques such as, for example, enclosing it in tablets. The bitter-tasting active compound is then not released in the mouth, and not until the gastrointestinal tract.

A further possibility of modification of the taste impression is the addition of taste modulators or flavors to the desired foods, drinks, articles consumed for pleasure, animal feed, sweeteners, cosmetics and pharmaceuticals.

It is therefore desirable to find substances which suppress or reduce the unpleasant taste impressions and also specifically amplify desired taste impressions, or at least do not adversely affect them.

In particular in the sector of pharmaceutical active compounds, a great number of substances which have in particular been bitterness modified are known. Thus, for example, the bitter taste of ibuprofen is masked by polylysine and polyarginine (cf. international patent application WO 2003/086293), by meglumine salt (cf. American patent U.S. Pat. No. 5,028,625), by sodium chloride or sodium saccharin (cf. international patent application WO 2003/0475550) or by hydroxypropyl-beta-cyclodextrin or chewable methacrylic acid copolymers (cf. Modifying Bitterness, Mechanism, Ingredients and Applications, Glenn Roy, 1997), in order to facilitate intake by patients. The bitterness of caffeine can also be reduced by a multiplicity of taste modulators such as, for example, by glutamic acid, dicalcium disalicylate, starch, lactose, mannitol and also by phosphatidic acid and beta-lactoglobulin (cf. Glenn Roy, 1997) and in addition by hydroxybenzamides, in particular hydroxybenzoic acid vanillylamide (cf. Ley et al., Journal of Agricultural & food Chemistry, 2006).

Further substances which have been used for reduction of a bitter taste in general and in particular in pharmaceuticals and foods are lecithin, ascorbate and citrate (cf. Japanese patent application JP 20011226293), esters of mono- or diglycerides such as glycerol monostearate and polycarboxylic acids such as succinic acid (cf. European patent application EP 0 732 064 A1), hydroxyflavanones (cf. European patent application EP 1 258 200 A1), 2-phenyl-4-chromanone derivatives (cf. German patent application DE 101 22 898), sodium sulfate hydrate (cf. Japanese patent application JP 02025428). In addition, American patent U.S. Pat. No. 5,637,618 discloses the use of benzoic acid derivatives for reduction of the bitter taste in drinks and also of sweeteners and of potassium chloride. The bitter taste of potassium chloride is also inhibited using 2,4-dihydroxybenzoic acid, carrageenan and thaumatin (cf. Glenn Roy, 1997; American patent U.S. Pat. No. 5,637,618, and also Japanese patent applications JP 04262758 and JP 07083684).

The known taste-modulators, however, are not completely satisfactory, in particular when the intention is to use them for reduction of the bitter taste of compositions of matter such, as for example, foods, drinks, articles consumed for pleasure, sweeteners, animal feed, cosmetics and pharmaceuticals which comprise at least one HIS, in particular HIS-comprising soft drinks. Their bitterness-reducing action is frequently insufficient. If, for this reason, the concentration of the known taste modulators is increased in order to achieve sufficient action, unwanted physiological or physical and/or chemical interactions with the remaining components of the respective compositions and/or adverse effects, in particular impairment up to complete distortion of their characteristic taste impression can occur.

### Object of the Invention

Accordingly the object of the present invention was to find a novel process for taste modulation, in particular for reduction of bitter taste and aftertaste, of compositions of matter which comprise at least one high intensity sweetener (HIS), in particular of foods, drinks, articles consumed for pleasure, sweeteners, animal feeds, cosmetics and pharmaceuticals which comprise at least one HIS.

The novel process for taste modulation is intended to have the effect that the taste modulators used do not cause any unwanted physiological or physical and/or chemical interactions with the remaining components of the respective compositions, in particular the foods, drinks, articles consumed for pleasure, sweeteners, animal feeds, cosmetics and pharmaceuticals, and the characteristic taste impression is not adversely affected, in particular is not impaired or completely distorted.

In particular, the novel process is intended to enable the bitter taste and the bitter aftertaste of compositions of matter which comprise at least one HIS to be significantly reduced.

### Solution According to the Invention

Accordingly, the novel process for taste modulation of compositions of matter which comprise at least one high intensity sweetener HIS has been found in which a polyoxyethylene fatty acid ester is added to a composition of matter which comprises at least one HIS.

Hereinafter the novel process for taste modulation of compositions of matter is termed “process according to the invention”.

### Advantages of the Invention

With respect to the prior art, it was surprising and not predictable by those skilled in the art that the object of the present invention could be achieved using the process according to the invention.

Surprisingly, the process according to the invention had the effect that the taste modulators used caused no unwanted physical and/or chemical interactions with the remaining components of the respective compositions of matter which comprised at least one HIS, in particular of the foods, drinks, articles consumed for pleasure, sweeteners, animal feeds, cosmetics and pharmaceuticals, and their characteristic taste impression was not adversely affected, in particular not impaired or even completely distorted.

In particular, the process according to the invention made it possible to reduce significantly, even with very low amounts of taste modulators, the bitter taste and bitter aftertaste of compositions of matter which comprised at least one HIS.

In particular, these surprisingly low amounts did not also adversely affect the color impression of the compositions of matter, which was a very particular advantage, in particular in the case of colored soft drinks.

It was especially surprising that the taste modulation of a given composition of matter which comprised at least one HIS was outstandingly reproducible by the process according to the invention, which was a very particular advantage precisely with respect to the production of mass market products such as foods, drinks, articles consumed for pleasure, sweeteners, animal feeds, cosmetics and pharmaceuticals.

### DETAILED DESCRIPTION OF THE INVENTION

The process according to the invention relates to the taste modulation, in particular reduction of the bitter taste and of the bitter aftertaste, of compositions of matter which comprise at least one high intensity sweetener (HIS).

Preferably, the compositions of matter are foods, drinks, articles consumed for pleasure, sweeteners, animal feeds, cosmetics and pharmaceuticals. Preferably, the drinks are soft drinks, particularly preferably caffeine-comprising soft drinks, in particular cola drinks.

The compositions of matter comprise at least one high intensity sweetener HIS as sweetener or sweetening agent. HIS is taken to mean compounds of synthetic or natural origin which have no physiological calorific value, or a physiological calorific value which is negligible in comparison to the sweetening strength (non-nutritive sweeteners), and have a sweetening strength many times higher than sucrose. The sweetening strength of a compound is given by the dilution at which it tastes just as sweet as a sucrose solution (isosweet solution; 0.1 M=4%), i.e. a 500-fold diluted solution of a sweetening agent has an isosweet taste to a sucrose solution when the sweetening agent has a sweetening strength of 500.

Examples of suitable HISs are known from Römpp Online 2007, “Süβstoffe” [Sweetening agents]. Preferably, the HISs are selected from the group consisting of acesulfame potassium (ACK), aspartame (ASP), saccharin (SAC) and salts thereof, cyclamate and salts thereof, aspartame-acesulfame salt, sucralose, thaumatin, stevia, stevioside and neohesperidine dihydrochalcone, preferably ACK, ASP, SAC and sucralose, particularly preferably ACK and SAC, in particular SAC.

Preferably, the composition of matter is a low-sugar composition which comprises less than 10 g, preferably less than 1 g, of sugar per liter or per kg of composition, in particular a sugar-free composition. Sugar in the present case is understood to mean, in particular, not exclusively mono- and disaccharides, however.

Advantageously, the composition of matter is a composition comprising less than 100 kJ, preferably less than 10 kJ, per liter or kg of composition.

Advantageously, the composition of matter is a carbohydrate-free, in particular starch-free, composition.

Preferably, the composition of matter is a low-fat composition which comprises less than 1 g of fat per liter or per kg of composition, in particular, a fat-free composition.

The low-sugar and/or low-fat compositions, in particular the sugar-free and/or fat-free compositions, are preferably an SAC-sweetened composition, in particular an SAC-sweetened drink.

The inventive fatty acid ester preferably comprises a mono- or trifatty acid ester, more preferably a monofatty acid ester.

The fatty acids may be either saturated or at least monounsaturated fatty acids. They are preferably saturated fatty acids.

The fatty acids of the inventive polyoxyethylene sorbitan fatty acid esters are, in one embodiment, C11 to C18 fatty acids, preference being given to selecting a fatty acid from the group consisting of lauric acid, oleic acid, palmitic acid and stearic acid. Very particular preference is given to using a palmitic ester, especially a polyoxyethylene sorbitan monopalmitic ester.

The inventive fatty acid esters are preferably the European Union approved food additives E432 (Polysorbate 20), E433 (Polysorbate 80), E434 (Polysorbate 40), E435 (Polysorbate 60) or E436 (Polysorbate 65), very particularly preferably E434.

In one embodiment of the process according to the invention, the at least one polyoxyethylene sorbitan monopalmitic ester is added in an amount such that, in the compositions of matter, a concentration per fatty acid ester of 1 to 100 mg, in particular 10 to 15 mg, per liter or kilogram of composition is ultimately present. Particularly preferably, the composition of matter comprises 13 mg of fatty acid ester, in particular of E434, per liter or kilogram of composition, preferably per liter of drink.

Very particular preference is given to a sugar-free composition, in particular a drink, which comprises SAC and a polyoxyethylene fatty acid ester, preferably E434.

In addition to the above-described polyoxyethylene fatty acid esters to be used according to the invention, other conventional and known taste-modulating substances can also be used in active amounts. Examples of suitable conventional and known taste-modulating substances are those described at the outset.

A further aspect of the invention is the use of at least one polyoxyethylene fatty acid ester as taste modulator in compositions of matter which comprise at least one high intensity sweetener (HIS). The polyoxyethylene fatty acid esters used in this case are advantageously the polyoxyethylene fatty acid esters and particular embodiments thereof which are described above in connection with the process according to the invention. With respect to the composition of matter, it is, in particular, the embodiments of the compositions of matter which are described in connection with the process according to the invention.

The embodiments which are described serve for illustration and for better understanding of the invention and are in no way to be understood as limiting. Further features of the invention are evident from the description hereinafter of preferred embodiments in combination with the subclaims. In this case the individual features of the invention can be implemented in one embodiment in each case individually or severally, and in no way represent a restriction of the invention to the described embodiment. The wording of the patent claims is hereby explicitly made subject matter of the description.

### Examples

### Example 1

The use of polyoxyethylene fatty acid ester for taste modulation of SAC For example 1, the following substances were used.

HIS:

Saccharin (SAC) from Sigma-Aldrich Chemie GmbH, Taufkirchen, Germany;

Polyoxyethylene Fatty Acid Ester:

Tween 40 (E434) from Sigma-Aldrich Chemie GmbH, Taufkirchen, Germany:

Quantitative Sensory Testing—General Test Protocol:

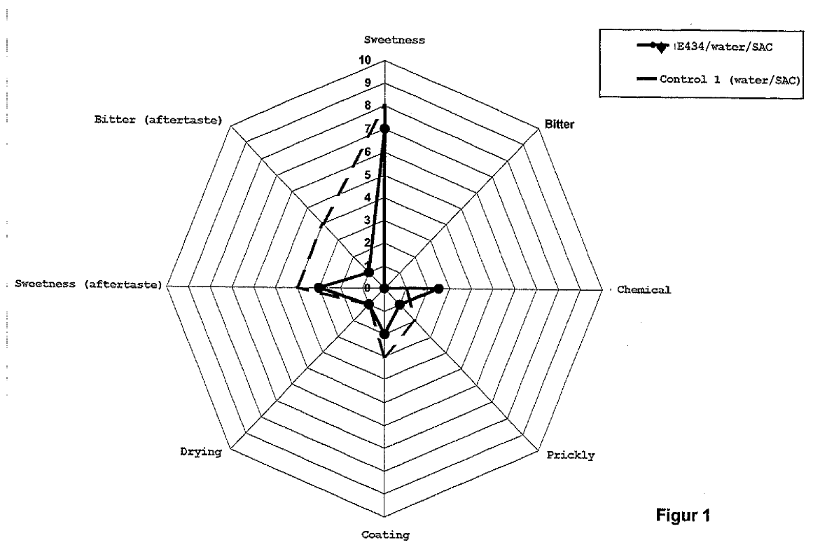

Consensus profiles of sample 1 of example 1, and also of the control sample 1, were established in agreement with DIN 10967-2/ISO 11035. For this, 8 trained testers who had been selected in accordance with the DIN/ISO protocols were familiarized with the product by definition and training of the predetermined feature properties. Then the testers tasted sample 1 for taste, aftertaste and mouthfeel in accordance with the predetermined feature properties. The respective consensus profiles were summarized by the test supervisor in the form of tables and what are termed spider plot diagrams (FIG. 1). Hereinafter, for the sake of clarity, the results are also given as a table.

Sample 1 and Control Sample 1—Composition of Matter:

Sample 1 and control sample 1 had the compositions of matter described hereinafter. The respective abbreviations which are used in the table below are given in brackets.

Control Sample 1:

Water+180 mg/l of SAC

(abbreviation: water/SAC)

Sample 1—Example 1:

Water+180 mg/l of SAC+13 mg/l of E434

(abbreviation: water/SAC/E434)

Sample 1 and Control Sample 1—Experimental Results:

The results of the quantitative sensory testing of sample 1 and of control sample 1 are summarized in table 1

In the table, the measured value 0 means that the relevant sensory property was not present, whereas the measured value 10 means that the relevant sensory property was strongly present.

TABLE 1
Quantitative sensory testing of sample 1 and control sample 1-
consensus profiles

| Control sample/ | Taste | | Mouthfeel | | | | Aftertaste | |
|---|---|---|---|---|---|---|---|---|
| sample | S^{a)} | Bt^{b)} | C^{c)} | K^{d)} | Bl^{e)} | A^{f)} | S(N)^{g)} | Bt(N)^{h)} |
| Control sample 1 | 8 | 0 | 1 | 2 | 3 | 1 | 4 | 4 |
| Water/SAC | | | | | | | | |
| Sample 1 | 7 | 0 | 2.5 | | 2 | 1 | 3 | 1 |
| Water/SAC/ | | | | | | | | |
| E434 | | | | | | | | |

^{a)}S = sweet;
^{b)}Bt = bitter;
^{c)}C = chemical;
^{d)}K = prickly;
^{e)}Bl = coating;
^{f)}A = drying;
^{g)}S(N) = sweet aftertaste;
^{h)}Bt(N) = bitter aftertaste;

The results of table 1 and of FIG. 1 prove that E434 had such a strong taste-modulating effect that the bitter aftertaste of SAC in water was very greatly reduced.

1.-33. (canceled)

34. A process for the taste modulation of compositions of matter which comprise as sweetener at least one high intensity sweetener HIS, wherein the HIS is saccharin (SAC), and wherein the bitter taste and/or aftertaste is reduced, the process comprising adding at least one polyoxyethylene sorbitan fatty acid ester which is suitable for food to the compositions of matter.

35. The process according to claim 34, wherein the ester comprises mono- and/or trifatty acid esters.

36. The process according to claim 34, wherein the ester comprises a monofatty acid ester.

37. The process according to claim 34, wherein the fatty acid is C11 to C18 fatty acid.

38. The process according to claim 34, wherein the fatty acid is a saturated or unsaturated fatty acid.

39. The process according to claim 34, wherein the fatty acid is selected from the group consisting of lauric acid, oleic acid, palmitic acid and stearic acid.

40. The process according to claim 34, wherein the fatty acid is palmitic acid.

41. The process according to claim 34, wherein the polyoxyethylene sorbitan fatty acid ester is polyoxyethylene sorbitan monopalmitic ester.

42. The process according to claim 34, wherein the at least one polyoxyethylene sorbitan fatty acid ester is added in an amount such that a concentration of 1 to 100 mg per liter or kg of composition results in the compositions of matter.

43. The process according to claim 34, wherein the compositions of matter are foods, drinks, articles consumed for pleasure, sweeteners, animal feeds, cosmetics or pharmaceuticals.

44. The process according to claim 34, wherein the compositions of matter are low-sugar compositions having less than 10 g of sugar per liter or kg of composition.

45. The process according to claim 34, wherein the composition is a low-carbohydrate.

46. The process according to claim 34, wherein the composition is a fat-free composition.

47. The process according to claim 34, wherein the composition of matter is a SAC-sweetened and sugar-free drink.

48. The process according to claim 34, wherein the at least one polyoxyethylene sorbitan fatty acid ester is added in an amount such that a concentration of 10 to 15 mg per liter or kg of composition results in the compositions of matter.

49. The process according to claim 34, wherein the at least one polyoxyethylene sorbitan fatty acid ester is added in an amount such that a concentration of 13 mg per liter or kg of composition results in the compositions of matter.

50. The process according to claim 34, wherein the compositions of matter are sugar-free compositions.

51. The process according to claim 34, wherein the composition of matter is an SAC-sweetened and sugar-free drink which comprises caffeine.

52. The process according to claim 34, wherein in the taste modulation the bitter taste and/or aftertaste of an SAC-sweetened and sugar-free drink is reduced.
